(19) United States
(12) Patent Application Publication
(10) Pub. No.: US 2011/0262453 A1
(43) Pub. Date: Oct. 27, 2011

(54) Markers for Assessing the Susceptibility of Cancer to IGF-1R Treatment

(76) Inventors: Samir E. Witta, Greenwood Village, CO (US); Koichi Yoshida, Tokyo (JP); Rafal Dziadziuszko, Gdarisk (PL); Fred R. Hirsch, Denver, CO (US)

(21) Appl. No.: 13/059,931

(22) PCT Filed: Aug. 20, 2009
(86) PCT No.: PCT/US09/54513

§ 371 Date: Jun. 9, 2011

Related U.S. Application Data
(60) Provisional application No. 61/090,387, filed on Aug. 20, 2008.

(51) Int. Cl. A61K 39/395; A61P 35/00; A61K 38/45; C12Q 1/02

(52) U.S. Cl. 424/158.1; 435/29; 424/94.5

(57) ABSTRACT

The present invention relates to a method of predicting response of a cancer cell tissue to IGF-IR treatment. The inventors have observed a direct relationship between the expression of survivin, ErbB3 and E-cadherin and the response of cancer cells to IGF-IR treatment. This observation permits screening of tumor cells prior to treatment to determine whether the cancer will respond to IGF-IR treatment, and/or to monitor the efficacy of such a treatment by conducting “before and after” assessments of the expression of these markers

The present application claims benefit of priority to U.S. Provisional Application Ser. No. 61/090,387, filed Aug. 20, 2008, the entire contents of which are hereby incorporated by reference.

### BACKGROUND OF THE INVENTION

I. Field of the Invention

The present invention relates generally to the fields of oncology and cancer therapy. More particularly, it concerns the assessment of biomarkers that predict the efficacy of an anti-cancer therapy.

II. Description of Related Art

The insulin-like growth factors, also known as somatomedins, include insulin-like growth factor-I (IGF-I) and insulin-like growth factor-II (IGF-II) (Klapper et al., 1983; Rinderknecht et al., 1978). These growth factors exert mitogenic activity on various cell types, including tumor cells (Macaulay, 1992), by binding to a common receptor named the insulin-like growth factor receptor-1 (IGF-1R) (Sepp-Lorenzino, 1998). Interaction of IGF's with IGF-1R activates the receptor by triggering autophosphorylation of the receptor on tyrosine residues (Butler et al., 1998). Once activated, IGF-1R in turn phosphorylates intracellular targets to activate cellular signaling pathways. This receptor activation is critical for stimulation of tumor cell growth and survival. Therefore, inhibition of IGF-1R activity represents a valuable potential method to treat or prevent growth of human cancers and other proliferative diseases.

Several lines of evidence indicate that IGF-I, IGF-II and their receptor IGF1R are important mediators of the malignant phenotype. Plasma levels of IGF-I have been found to be the strongest predictor of prostate cancer risk (Chan et al., 1998) and similar epidemiological studies strongly link plasma IGF-I levels with breast, colon and lung cancer risk. Overexpression of IGF-1R has also been demonstrated in several cancer cell lines and tumor tissues. IGF1R is overexpressed in 40% of all breast cancer cell lines (Pandini et al., 1999) and in 15% of lung cancer cell lines. In breast cancer tumor tissue, IGF1R is overexpressed 6-14 fold and IGF1R exhibits 2-4 fold higher kinase activity as compared to normal tissue (Webster et al., 1996; Pekonen et al., 1998). Ninety percent of colorectal cancer tissue biopsies exhibit elevated IGF-1R levels wherein the extent of IGF-1R expression is correlated with the severity of the disease. Analysis of primary cervical cancer cell cultures and cervical cancer cell lines revealed 3- and 5-fold overexpression of IGF-1R, respectively, as compared to normal ectocervical cells (Steller, et al., 1996). Expression of IGF-1R in synovial sarcoma cells also correlated with an aggressive phenotype (i.e., metastasis and high rate of proliferation; Xie et al., 1999).

Currently, there are several known anti-cancer therapies that target IGF-1R and decrease IGF-1R function and/or expression are effective in the treatment of some cancer patients. However, it is expected that a portion of cancer patients may not respond to such treatments. At least one earlier attempt has been made along these lines, by selecting a patient or patient population having a tumor known to express (i) IRS-1 phosphorylation on tyrosine 896; (ii) IRS-1 phosphorylation on tyrosine 612; (iii) IRS-1 phosphorylation on any tyrosine; (iv) IGF-II; (v) IGF1R phosphorylation on any tyrosine; or (vi) IGF1R (see U.S. Patent Publication 2006/0140960). Nonetheless, there remains a need in the art for improved methods of identifying specific cancer populations and/or specific cancer patients who are most likely to respond to one or more anti-cancer therapies that target IGF-1R.

### SUMMARY OF THE INVENTION

Thus, in accordance with the present invention, there is provided a method of predicting response of a cancer cell tissue to IGF-1R treatment comprising (a) assessing the expression of one or more of survivin, ErbB3 and E-cadherin in said cancer cell tissue; and (b) predicting a favorable response if one or more of survivin, ErbB3 and E-cadherin is detected in said cancer cell tissue. The method may assess the expression of two or more of survivin, ErbB3 and E-cadherin, or all three of survivin, ErbB3 and E-cadherin. The method may further comprise assessing the expression of IGF-1R in said cancer cell tissue. The expression of one, two or all three of survivin, ErbB3 and E-cadherin may be elevated with respect to average expression in a normal population.

The method may further comprise treating said cancer cell tissue with an inhibitor of IGF-1R, such as TKI, or an anti-IGF-1R antibody. The method may further comprise, following treating, assessing the expression of one or more of survivin, ErbB3 and E-cadherin in said cancer cell tissue. The method may further comprise obtaining said cancer cell tissue by biopsy of a tumor from a subject. The method may also further comprise making a treatment decision for said subject based on the outcome of step (b). Assessing may comprise a nucleic acid based assay, including RT-PCR, or may comprise immunologic analysis. The cancer tissue may be lung cancer tissue, prostate cancer tissue, pancreatic cancer tissue, gastric cancer tissue, esophageal cancer tissue, colon cancer tissue or breast cancer tissue, including non-small cell lung cancer tissue.

In another embodiment, there is provided a method of predicting outcome of an IGF-1R cancer treatment in a subject comprising (a) assessing the expression of one or more of survivin, ErbB3 and E-cadherin in cancer cells from said subject; (b) treating a subject with an IGF-1R cancer treatment if expression of one or more of survivin, ErbB3 and E-cadherin is detected, or treating a subject with a non-IGF-1R cancer treatment if expression of survivin, ErbB3 or E-cadherin is not detected. The method may assess the expression of two or more of survivin, ErbB3 and E-cadherin, or all three of survivin, ErbB3 and E-cadherin. The method may further comprise assessing the expression of IGF-1R in said cancer cell tissue. The expression of one, two or all three of survivin, ErbB3 and E-cadherin may be elevated with respect to average expression in a normal population. The subject may be a human subject. The IGF-1R cancer treatment may be TKI, or an anti-IGF-1R antibody. The non-IGF-1R cancer treatment may comprise chemotherapy, radiotherapy and/or surgery.

The method may further comprise obtaining said cancer cells by a first biopsy of a tumor from said subject. The method may further comprising obtaining, after step (b), additional cancer cells by a second biopsy of said tumor from said subject, and assessing the expression of one or more of survivin, ErbB3 and E-cadherin in cancer cells of said second biopsy. Assessing may comprise a nucleic acid based assay, including RT-PCR, or may comprise immunologic analysis. The cancer tissue may be lung cancer tissue, prostate cancer tissue, pancreatic cancer tissue, gastric cancer tissue, esophageal cancer tissue, colon cancer tissue or breast cancer tissue, including non-small cell lung cancer tissue.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### DETAILED DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

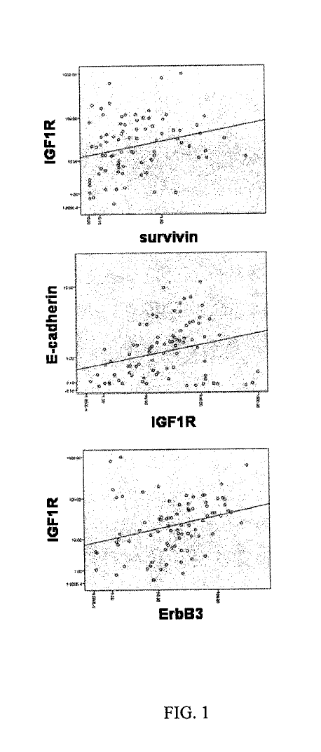

FIG. 1—Gene expression in primary tumors from patients with NSCLC. Primary tumors were evaluated using real time RT-PCR and normalized to internal standard, β-actin. R and p values are depicted in the Table 2, below.

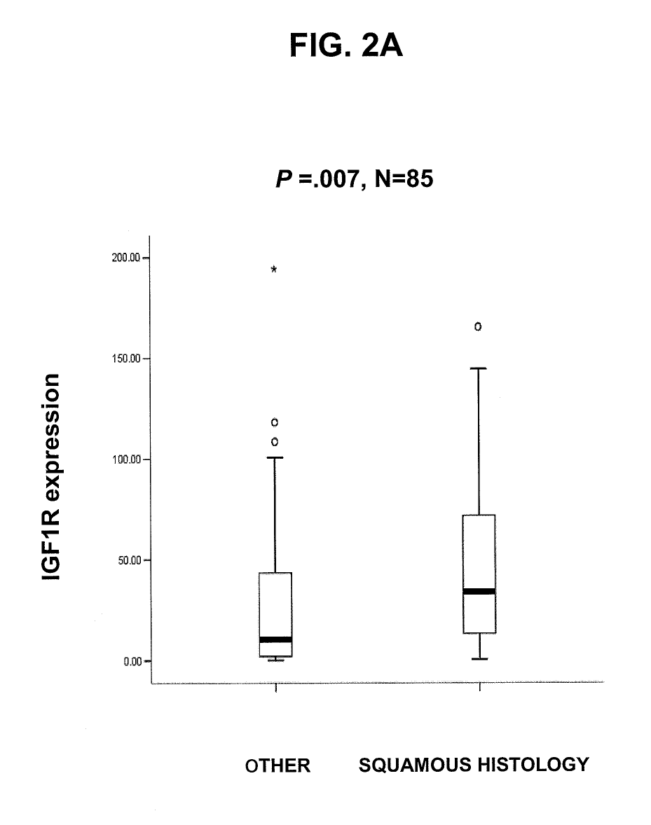

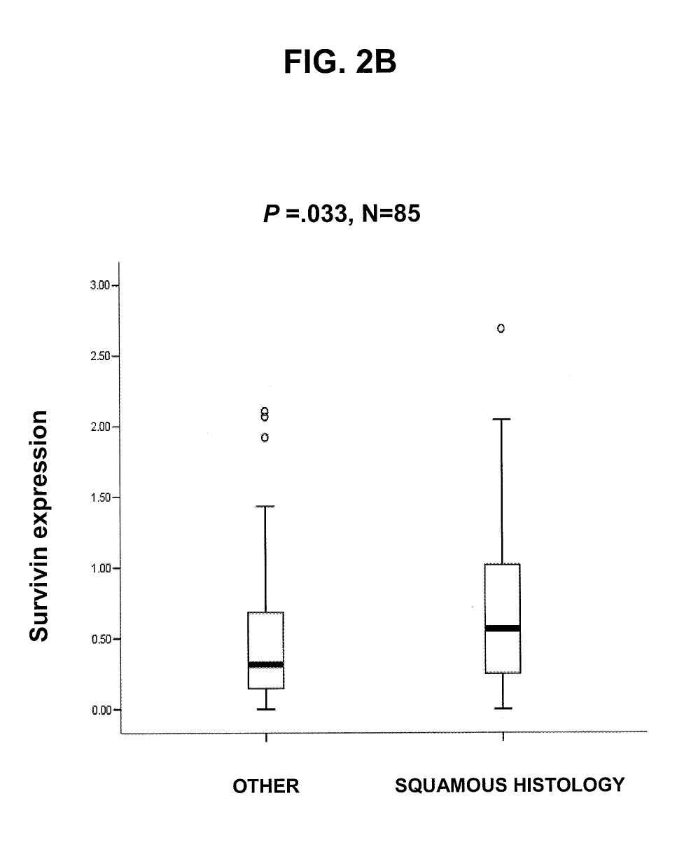

FIG. 2A—IGF-1R has higher expression in patients with SCC histology.

FIG. 2B—Survivin has higher expression in patients with SCC histology.

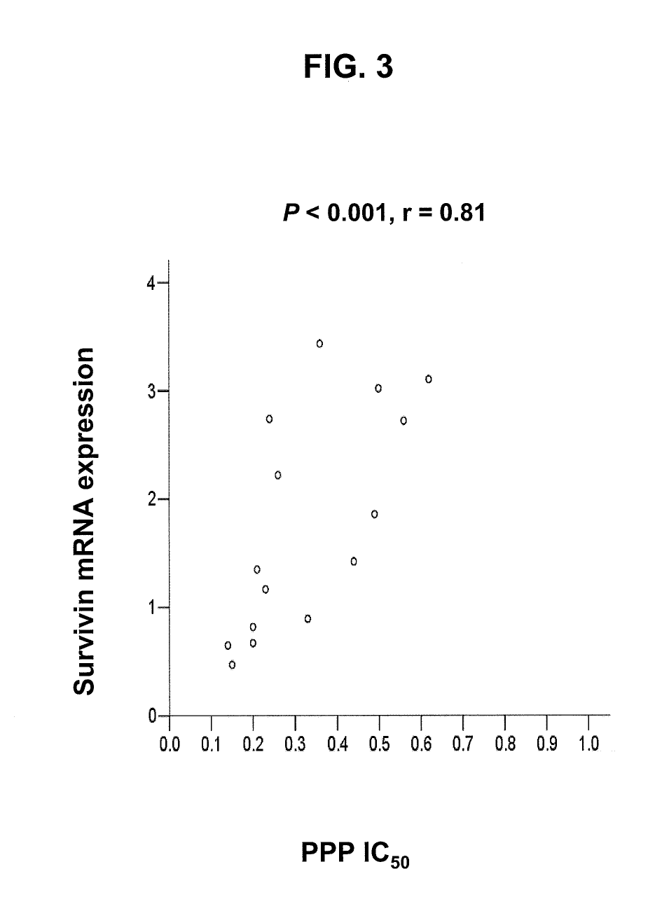

FIG. 3—Survivin expression associates with activity of the IGF-1R TKI, PPP.

### DESCRIPTION OF THE ILLUSTRATIVE EMBODIMENTS

### I. THE PRESENT INVENTION

During the last decade, several pathways were identified as targets for novel treatments for lung cancer. Among those is IGFR-1 and its downstream targets. IGFR-1 is a transmembrane heterotetrameric protein, encoded by the gene IGF1R gene located on chromosome 15q25-q26, implicated in promoting oncogenic transformation, growth and survival of cancer cells. Activation of IGF1R leads to the alterations in the Ras/Raf/mitogen-activated protein kinase, and phosphoinositol-3-kinase (PI3K) signaling. These events lead to increased expression of survivin and on the other hand, inhibition of IGF1r leads to downregulation of survivin. Previous studies indicated that IGFR-1 expression and activation have been linked to disease progression, increased resistance to radiotherapy and poor prognosis. IGFR-1 is suggested to implicated in resistance to other targeted agents, such as EGFR inhibitors in NSCLC.

This work led to the development of several molecules, tyrosine kinase inhibitors and antibodies, targeting IGF1R. These agents age being evaluated in Phase I-III clinical trials. In those trials, it was observed that higher responses were detected in patients with squamous cell carcinoma, which led to the design of phase III clinical trials enrolling only patients with squamous cell carcinoma histology. Recent studies showed that molecules involved in epithelial-mesenchymal transformation (EMT) influence response to targeted therapies in cancer, but the relationship between response and target characteristics remains incompletely understood. Hence, there is a need to properly identify which cancers are susceptible to treatment with agents that inhibit IGF-1R, thereby avoiding the time, expense and complications of administering a therapy that will not be effective.

Here, the inventors correlate the expression of Survivin and several other molecules involved in EMT with expression of IGF-1R in primary tumors from patients with NSCLC and secifically in patients with SCC. They also correlate the expression of survivin with the activity of survivin in NSCLC cell lines. Finally, the results presented show that IGF-1R is correlated with and Survivin expression, E-cadherin expression, and ErbB3 expression in 85 patients with NSCLC. Thus, these findings provide targets that can be assayed in a non-invasive fashion to determine the efficacy of IGR-1R therapies.

### II. CANCER, IGF-1R AND TARGETED TREATMENTS

A. Cancers

Exemplary cancers for which treatment is contemplated in the present invention include lung cancer (including non-small cell lung cancer), esophageal cancer, prostate cancer, pancreatic cancer, gastric cancer, and breast cancer.

The cancer may be treated after its initial diagnosis or subsequently by therapies or combination of two or more therapies. A cancer recurrence may be defined as the reappearance or rediagnosis of a patient as having any cancer following any treatment including one or more of surgery, radiotherapy or chemotherapy. The patient with relapsed disease need not have been reported as disease free, but merely that the patient has exhibited renewed cancer growth following some degree of clinical response by the first therapy.

The therapy or clinical response may result in, but is not limited to, stable disease, tumor regression, tumor necrosis, absence of demonstrable cancer, reduction in tumor size or burden, blocking of tumor growth, reduction in tumor-associated pain, reduction in tumor associated pathology, reduction in tumor associated symptoms, tumor non-progression, increased disease free interval, increased time to progression, induction of remission, reduction of metastasis, or increased patient survival.

B. IGF-1R

Insulin-like Growth Factor 1 Receptor (IGF-1R) is a transmembrane receptor that is activated by IGF-1 and by the related growth factor IGF-2. It belongs to the large class of tyrosine kinase receptors. This receptor mediates the effects of IGF-1, which is a polypeptide protein hormone similar in molecular structure to insulin. IGF-1 plays an important role in growth and continues to have anabolic effects in adults—meaning that it can induce hypertrophy of skeletal muscle and other target tissues. Mice lacking the IGF-1 receptor die late in development, and show a dramatic reduction in body mass, testifying to the strong growth-promoting effect of this receptor. Mice carrying only one functional copy of igflr are normal, but exhibit a ˜15% decrease in body mass.

Two α subunits and two β subunits make up the IGF-1 receptor. The β subunits pass through the cellular membrane and are linked by disulfide bonds. The receptor is a member of a family which consists of the Insulin Receptor and the IGF-2R (and their respective ligands IGF-1 and IGF-2), along with several IGF-binding proteins. IGF-1R and IR both have a binding site for ATP, which is used to provide the phoshates for autophosphorylation. There is a 60% homology between IGF-1R and the insulin receptor.

Tyrosine kinase receptors, including, the IGF-1 receptor, mediate their activity by causing the addition of a phosphate groups to particular tyrosines on certain proteins within a cell. This addition of phosphate induces what are called “cell signaling” cascades—and the usual result of activation of the IGF-1 receptor is survival and proliferation in mitosis-competent cells, and growth (hypertrophy) in tissues such as skeletal muscle and cardiac muscle. During embryonic development, the IGF-1R pathway is involved with the developing limb buds.

The IGFR signalling pathway is of critical importance during normal development of mammary gland tissue during pregnancy and lactation. During pregnancy, there is intense proliferation of epithelial cells which form the duct and gland tissue. Following weaning, the cells undergo apoptosis and all the tissue is destroyed. Several growth factors and hormones are involved in this overall process, and IGF-1R is believed to have roles in the differentiation of the cells and a key role in inhibiting apoptosis until weaning is complete.

The IGF-1R is implicated in several cancers, most notably breast cancer. In some instances its anti-apoptotic properties allow cancerous cells to resist the cytotoxic properties of chemotheraputic drugs or radiotherapy. In others, where EGFR inhibitors such as erlotinib are being used to inhibit the EGFR signalling pathway, IGF-1R confers resistance by forming one half of a heterodimer (see the description of EGFR signal transduction in the erlotinib page), allowing EGFR signalling to resume in the presence of a suitable inhibitor. This process is referred to as crosstalk between EGFR and IGF-1R. It is further implicated in breast cancer by increasing the metastatic potential of the original tumour by inferring the ability to promote vascularisation.

C. IGF-1R Targeted Treatments

Currently, there are several known anti-cancer therapies that target IGF1R. For example, anti-IGF1R antibodies are disclosed by Schering Corp (see WO 2003/100008); Pfizer (see WO 2002/53596 or WO 2004/71529); Pierre Fabre medicament (see WO 2003/59951), Pharmacia Corp. (see WO 2004/83248), Immunogen, Inc. (see WO 2003/106621), Hoffman La Roche (see WO 2004/87756) and Imclone Systems Inc. (IMC-A12; see Burtrum et al., 2003). Additionally, Novartis has described a small molecule IGFR inhibitor, NVP-ADW-742 (see WO 2002/92599) as has Biotech Research Ventures PTE Ltd (see WO 2003/39538). Antisense Therapeutics Ltd. has described an anti-sense therapy that inhibits IGF 1 R expression, ATL-1101.

Due to the similarity of the structures of IGF-1R and the insulin receptor, especially in the regions of the ATP binding site and tyrosine kinase regions, synthesizing selective inhibitors of IGF-1R is difficult. The cyclolignan picropodophyllin (PPP) is a cis-isomer of podophyllotoxin. Girnita et al. (2004) were the first to report the potent and selective action of this agent against IGF-1R. They showed that this compound, which is almost nontoxic (LD₅₀>500 mg/kg in rodents), efficiently blocked IGF-1R activity, reduced pAkt and phosphorylated extracellular signal regulated kinase 1 and 2 (pErk1/2), induced apoptosis in cultured IGF-1R-positive tumor cells, and caused complete tumor regression in xenografted and allografted mice. The authors suggested that PPP or related compounds with inhibitory effects on IGF-1R should be considered in the development of anti-cancer agents. Other particular inhibitors include tyrphostins such as AG538 and AG1024, which show some selectivity towards IGF-1R over IR; pyrrolo[2,3-d]-pyrimidine derivatives such as NVP-AEW541, which show far greater (100-fold) selectivity towards IGF-1R over IR. Another IGF-1R therapy is PQIP.

As mentioned above, another class of IGR-1R-targeted therapies include antibodies to IGF-1R, such as MK-0646, AMB 479, AVE1642. See also U.S. Pat. No. 7,371,378; U.S. Patent Publications 2008/0181891, 2008/0152649, 2008/00669539 and 2007/0243194, each of which are incorporated by reference.

### III. TARGET GENES

As discussed above, the present invention involves the assessment of expression levels of three particular target genes that have been found to be linked to a cancer's suspectibility to treatment is IGF-1R inhibitors. These genes—survivin, ErbB3 and E-cadherin, are discussed below.

A. Survivin

Survivin, also called Baculoviral IAP repeat-containing 5 (BIRC5), is a human gene that is part of the inhibitor of apoptosis family (IAP). The survivin protein functions to inhibit caspase activation therefore leading to negative regulation of apoptosis or programmed cell death. This has been shown by disruption of survivin induction pathways leading to increase in apoptosis and decrease in tumour growth. The survivin protein is expressed highly in most human tumours and fetal tissue, but is completely absent in terminally differentiated cells. This fact therefore makes survivin an ideal target for cancer therapy as cancer cells are targeted while normal cells are left alone. Survivin expression is also highly regulated by the cell cycle and is only expressed in the G2-M phase. It is known that survivin localizes to the mitotic spindle by interaction with tubulin during mitosis and may play a contributing role in regulating mitosis. The molecular mechanisms of survivin regulation are still not well understood to this day, but reguation of survivin seems to be linked to the p53 protein.

A structural feature common to all IAP family proteins is that they all contain at least one baculoviral IAP repeat (BIR) domain characterized by a conserved zinc-coordinating Cys/His motif at the N-terminal half of the protein. Survivin is distinguished from other IAP family members in that it has only one BIR domain. The mice and human BIR domain of survivin are very similar structurally except for two differences which may affect function variability. The human survivin also contains an elongated C-terminal helix comprising 42 amino acids. Survivin is 16.5 kDa large and is the smallest member of the IAP family. The accession number for the mRNA is NM 001012270.

B. ErbB3

V-erb-b2 erythroblastic leukemia viral oncogene homolog 3 (avian), also known as ERBB3, is a human gene. This gene encodes a member of the epidermal growth factor receptor (EGFR) family of receptor tyrosine kinases. This membrane-bound protein has a neuregulin binding domain but not an active kinase domain. Thus, it can bind this ligand but not convey the signal into the cell through protein phosphorylation. However, it does form heterodimers with other EGF receptor family members which do have kinase activity. Heterodimerization leads to the activation of pathways which lead to cell proliferation or differentiation. Amplification of this gene and/or overexpression of its protein have been reported in numerous cancers, including prostate, bladder, and breast tumors. Alternate transcriptional splice variants encoding different isoforms have been characterized. One isoform lacks the intermembrane region and is secreted outside the cell. This form acts to modulate the activity of the membrane-bound form. Additional splice variants have also been reported, but they have not been thoroughly characterized. It is thought that ERBB3, when activated, becomes a substrate for dimerization and subsequent phosphorylation by ERBB1, ERBB2 and ERBB4. Like many of the receptor tyrosine-kinases, ERBB3 is activated by extracellular ligand. Ligands known to bind to ERBB3 include heregulin. The accession no. for ErbB3 mRNA is NM 001005915.

C. E-Cadherin

Cadherins are a class of type-1 transmembrane proteins. They play important roles in cell adhesion, ensuring that cells within tissues are bound together. They are dependent on calcium (Ca²⁺) ions to function, hence their name. The cadherin superfamily includes cadherins, protocadherins, desmogleins, and desmocollins, and more. In structure, they share cadherin repeats, which are the extracellular Ca²⁺-binding domains. There are multiple classes of cadherin molecule, each designated with a one-letter prefix (generally noting the type of tissue with which it is associated). Cadherins within one class will bind only to themselves. For example, an N-cadherin will bind only to another N-cadherin molecule. Because of this specificity, groups of cells that express the same type of cadherin molecule tend to cluster together during development, whereas cells expressing different types of cadherin molecules tend to separate. Different members of the cadherin family are found in different locations. E-cadherins are found in epithelial tissue; N-cadherins are found in neurons; and P-cadherins are found in the placenta. T-cadherins have no cytoplasmic domains and must be tethered to the plasma membrane. The accession no. for E-cadherin mRNA is NM 004360.

E-cadherin (epithelial) is probably the best understood cadherin. It consists of 5 cadherin repeats (EC1˜EC5) in the extracellular domain, one transmembrane domain, and an intracellular domain that binds p120-catenin and β-catenin. The intracellular domain contains a highly-phosphorylated region vital to β-catenin binding and therefore to E-cadherin function. β-catenin can also bind to α-catenin. α-catenin participates in regulation of actin-containing cytoskeletal filaments. In epithelial cells, E-cadherin-containing cell-to-cell junctions are often adjacent to actin-containing filaments of the cytoskeleton.

E-cadherin is first expressed in the 2-cell stage of mammalian development, and becomes phosphorylated by the 8-cell stage, where it causes compaction. In adult tissues, E-cadherin is expressed in epithelial tissues, where it is constantly regenerated with a 5-hour half-life on the cell surface. Loss of E-cadherin function or expression has been implicated in cancer progression and metastasis. E-cadherin downregulation decreases the strength of cellular adhesion within a tissue, resulting in an increase in cellular motility. This in turn may allow cancer cells to cross the basement membrane and invade surrounding tissues.

### IV. ASSESSMENT OF TARGET GENE EXPRESSION

Assessment of expression levels of the markers discussed above may be direct, as in the use of quantitative immunohistochemistry (IHC) or other antibody-based assays (Western blot, FIA, FISH, radioimmunoassay (RIA), RIP, ELISA, immunoassay, immunoradiometric assay, a fluoroimmunoassay, an immunoassay, a chemiluminescent assay, a bioluminescent assay, a gel electrophoresis), or indirectly by quantitating the transcripts for these genes (in situ hybridization, nuclease protection, Northern blot or PCR, including RT-PCR). Relevant methodologies are discussed below.

A. Nucleic Acid-Based Diagnosis

The present invention also comprises methods of examining mRNA expression as a measure of target protein levels. mRNA is isolated from cancer cells according to standard methodologies (Sambrook et al., 1989). It may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

A variety of different assays are contemplated, including but not limited to, fluorescent in situ hybridization (FISH), Northern blotting, dot blot analysis, and of course PCR and RT-PCR.

1. Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process. In particular embodiments, the probes or primers are labeled with radioactive species (³²P, ¹⁴C, ³⁵S, ³H, or other label), with a fluorophore (rhodamine, fluorescein) or a chemillumiscent (luciferase).

2. Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention, Walker et al., (1992).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double-stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single-stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR™” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide,” thereby amplifying the di-oligonucleotide, may also be used in the amplification step of the present invention. Wu et al., (1989), incorporated herein by reference in its entirety.

Real-time polymerase chain reaction, also called quantitative real time polymerase chain reaction (qPCR) or kinetic polymerase chain reaction, is a laboratory technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample.

The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce when hybridized with a complementary DNA.

Frequently, real-time polymerase chain reaction is combined with reverse transcription polymerase chain reaction to quantify low abundance messenger RNA (mRNA), enabling a researcher to quantify relative gene expression at a particular time, or in a particular cell or tissue type. Although real-time quantitative polymerase chain reaction is often marketed as RT-PCR, it should not be confused with reverse transcription polymerase chain reaction, also known as RT-PCR.

A DNA-binding dye binds to all double-stranded (ds)DNA in a PCR reaction, causing fluorescence of the dye. An increase in DNA product during PCR therefore leads to an increase in fluorescence intensity and is measured at each cycle, thus allowing DNA concentrations to be quantified. However, dsDNA dyes such as SYBR Green will bind to all dsDNA PCR products, including non-specific PCR products (such as “primer dimers”). This can potentially interfere with or prevent accurate quantification of the intended target sequence. The reaction is prepared as usual, with the addition of fluorescent dsDNA dye.

The reaction is run in a thermocycler, and after each cycle, the levels of fluorescence are measured with a detector; the dye only fluoresces when bound to the dsDNA (i.e., the PCR product). With reference to a standard dilution, the dsDNA concentration in the PCR can be determined.

Like other real-time PCR methods, the values obtained do not have absolute units associated with it (i.e. mRNA copies/cell). As described above, a comparison of a measured DNA/RNA sample to a standard dilution will only give a fraction or ratio of the sample relative to the standard, allowing only relative comparisons between different tissues or experimental conditions. To ensure accuracy in the quantification, it is usually necessary to normalize expression of a target gene to a stably expressed gene. This can correct possible differences in RNA quantity or quality across experimental samples.

Using fluorescent reporter probes is the most accurate and most reliable of the methods, but also the most expensive. It uses a sequence-specific RNA or DNA-based probe to quantify only the DNA containing the probe sequence; therefore, use of the reporter probe significantly increases specificity, and allows quantification even in the presence of some non-specific DNA amplification. This potentially allows for multiplexing—assaying for several genes in the same reaction by using specific probes with different-coloured labels, provided that all genes are amplified with similar efficiency.

It is commonly carried out with an RNA-based probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the taq polymerase breaks the reporter-quencher proximity and thus allows unquenched emission of fluorescence, which can be detected. An increase in the product targeted by the reporter probe at each PCR cycle therefore causes a proportional increase in fluorescence due to the breakdown of the probe and release of the reporter.

The PCR reaction is prepared as usual (see PCR), and the reporter probe is added. As the reaction commences, during the annealing stage of the PCR both probe and primers anneal to the DNA target. Polymerisation of a new DNA strand is initiated from the primers, and once the polymerase reaches the probe, its 5′-3-exonuclease degrades the probe, physically separating the fluorescent reporter from the quencher, resulting in an increase in fluorescence.

Fluorescence is detected and measured in the real-time PCR thermocycler, and its geometric increase corresponding to exponential increase of the product is used to determine the threshold cycle (C_{T}) in each reaction.

Quantitating gene expression by traditional methods presents several problems. Firstly, detection of mRNA on a Northern blot or PCR products on a gel or Southern blot is time-consuming and does not allow precise quantitation. Also, over the 20-40 cycles of a typical PCR reaction, the amount of product reaches a plateau determined more by the amount of primers in the reaction mix than by the input template/sample.

Relative concentrations of DNA present during the exponential phase of the reaction are determined by plotting fluorescence against cycle number on a logarithmic scale (so an exponentially increasing quantity will give a straight line). A threshold for detection of fluorescence above background is determined. The cycle at which the fluorescence from a sample crosses the threshold is called the cycle threshold, Cₜ. Since the quantity of DNA doubles every cycle during the exponential phase, relative amounts of DNA can be calculated, e.g., a sample whose Cₜ is 3 cycles earlier than another's has 2³=8 times more template.

Amounts of RNA or DNA are then determined by comparing the results to a standard curve produced by RT-PCR of serial dilutions (e.g., undiluted, 1:4, 1:16, 1:64) of a known amount of RNA or DNA. As mentioned above, to accurately quantify gene expression, the measured amount of RNA from the gene of interest is divided by the amount of RNA from a housekeeping gene measured in the same sample to normalize for possible variation in the amount and quality of RNA between different samples. This normalization permits accurate comparison of expression of the gene of interest between different samples, provided that the expression of the reference (housekeeping) gene used in the normalization is very similar across all the samples. Choosing a reference gene fulfilling this criterion is therefore of high importance, and often challenging, because only very few genes show equal levels of expression across a range of different conditions or tissues.

3. Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

4. Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al. (1989).

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

5. Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences that may then be analyzed by direct sequencing.

6. Kit Components

All the essential materials and reagents required for detecting and sequencing the gene of interest may be assembled together in a kit. This generally will comprise preselected primers and probes. Also included may be enzymes suitable for amplifying nucleic acids including various polymerases (RT, Taq, Sequenase™, etc.), deoxynucleotides and buffers to provide the necessary reaction mixture for amplification. Such kits also generally will comprise, in suitable means, distinct containers for each individual reagent and enzyme as well as for each primer or probe.

7. Chip Technologies

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al. (1994); Fodor et al. (1991).

B. Immunodiagnostics

Antibodies of the present invention can be used in characterizing the protein content of target cells through techniques such as ELISAs and Western blotting. This may provide a screen for the presence or absence of malignancy or as a predictor of future cancer or in the present case as a strategy to predict likely responses to exposure to an oncolytic virus.

The use of antibodies of the present invention, in an ELISA assay is contemplated. For example, anti-tumor suppressor antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for a tumor suppressor that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

Antibodies can also find use in immunoblots or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the tumor suppressor are considered to be of particular use in this regard.

### V. ALTERNATIVE CANCER THERAPIES

In accordance with certain embodiments of the present invention, applicants also provide methods for cancer treatment that do not involve the use of IGF-1R inhnibitors. Such therapies may be utilized when the assays of the present invention indicate that IGF-1R inhibitors will not be effective. They may also be used in combination therapy with IGF-1R inhibitors where the present invention indicates that IGR-1R therapy is appropriate.

A. Therapeutic Nucleic Acids

A “therapeutic nucleic acid” is defined herein to refer to a nucleic acid which can be administered to a subject for the purpose of treating or preventing a disease. The nucleic acid herein is one which is known or suspected to be of benefit in the treatment of a hyperproliferative disease. Therapeutic benefit may arise, for example, as a result of alteration of expression of a particular gene or genes by the nucleic acid. Alteration of expression of a particular gene or genes may be inhibition or augmentation of expression of a particular gene. Certain embodiments of the present invention concern the administration of a therapeutic nucleic acid.

The term “gene therapy” within this application can be defined as delivery of a therapeutic gene or other therapeutic nucleic acid to a patient in need of such for purposes of treating a hyperproliferative disease or for treating a condition which, if left untreated may result in a hyperproliferative disease. Encompassed within the definition of “therapeutic gene” is a “biologically functional equivalent” therapeutic gene. Accordingly, sequences that have about 70% to about 99% homology of amino acids that are identical or functionally equivalent to the amino acids of the therapeutic gene will be sequences that are biologically functional equivalents provided the biological activity of the protein is maintained. Classes of therapeutic genes include tumor suppressor genes, cell cycle regulators, pro-apoptotic genes, cytokines, toxins, anti-angiogenic factors, and molecules than inhibit oncogenes, pro-angiogenic factors, growth factors, antisense transcripts, ribozymes and RNAi.

Examples of therapeutic genes include, but are not limited to, p53, Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, Rb, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

Other examples of therapeutic genes include genes encoding enzymes. Examples include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidase, a pullanase, a recombinase, a reverse transcriptase, a topoisomerase, a xylanase, a reporter gene, an interleukin, or a cytokine.

Further examples of therapeutic genes include the gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione β-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, β-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase.

Therapeutic genes also include genes encoding hormones. Examples include, but are not limited to, genes encoding growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, α melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

Other examples of therapeutic genes include genes encoding antigens present in hyperproliferative tissues that can be used to elicit and immune response against that tissue. Anti-cancer immune therapies are well known in the art, for example, in greater detail in PCT application WO0333029, WO0208436, WO0231168, and WO0285287, each of which is specifically incorporated by reference in its entirety.

Yet other therapeutic genes are those that encode inhibitory molecules, such as antisense, ribozymes, siRNA and single chain antibodies. Such molecules can be used advantageously to inhibit hyperproliferative genes, such as oncogenes, inducers of cellular proliferation and pro-angiogenic factors.

B. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Intratumoral injection prior to surgery or upon excision of part of all of cancerous cells, tissue, or tumor, a cavity may be formed in the body. Treatment may be accomplished by perfusion, direct injection or local application of these areas with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

C. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

1. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents, some of which are discussed below.

2. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the syntheisis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

3. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

4. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

5. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

6. Other Agents

Other agents that may be used include bevacizumab (brand name Avastin®), gefitinib (Iressa®), trastuzumab (Herceptin®), cetuximab (Erbitux®), panitumumab (Vectibix®), bortezomib (Velcade®), and Gleevec. In addition, growth factor inhibitors and small molecule kinase inhibitors have utility in the present invention as well. All therapies described in Cancer: Principles and Practice of Oncology (7^{th} Ed.), 2004, and Clinical Oncology (3^{rd} Ed., 2004) are hereby incorporated by reference. The following additional therapies are encompassed, as well.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with p53 gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

Tumor Necrosis Factor is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

The use of sex hormones according to the methods described herein in the treatment of cancer. While the methods described herein are not limited to the treatment of a specific cancer, this use of hormones has benefits with respect to cancers of the breast, prostate, and endometrial (lining of the uterus). Examples of these hormones are estrogens, anti-estrogens, progesterones, and androgens.

Corticosteroid hormones are useful in treating some types of cancer (lymphoma, leukemias, and multiple myeloma). Corticosteroid hormones can increase the effectiveness of other chemotherapy agents, and consequently, they are frequently used in combination treatments. Prednisone and dexamethasone are examples of corticosteroid hormones.

D. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques. Stereotactic radiotherapy is used to treat brain tumors. This technique directs the radiotherapy from many different angles so that the dose going to the tumor is very high and the dose affecting surrounding healthy tissue is very low. Before treatment, several scans are analyzed by computers to ensure that the radiotherapy is precisely targeted, and the patient's head is held still in a specially made frame while receiving radiotherapy. Several doses are given.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment you will have a specially made metal frame attached to your head. Then several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

E. Combination Therapies

Any of the foregoing therapies may be combined with IGF-1R therapies to increase the killing of cancer cells, the inhibition of cancer cell growth, the inhibition of metastasis, the inhibition of angiogenesis or otherwise improve the reverse or reduction of malignant phenotype of tumor cells. These compositions would be provided in a combined amount effective to kill or inhibit proliferation of the cell. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent.

Alternatively, the gene therapy treatment may precede or follow the other agent treatment by intervals ranging from minutes to weeks. In embodiments where the agents are applied separately to the cell, one would generally ensure that a significant period of time did not expire between the time of each delivery, such that the agents would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either agent will be desired. Various combinations may be employed, where the IGF-1R therapy is “A” and the other agent is “B,” as exemplified below:

| | | | | | | | |
|---|---|---|---|---|---|---|---|
| A/B/A | B/A/B | B/B/A | A/A/B | B/A/A | A/B/B | B/B/B/A | B/B/A/B |

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are contemplated. Again, to achieve cell killing, both agents are delivered to a cell in a combined amount effective to kill the cell.

### VI. EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Patient population. The cohort consisted of sequential 85 patients who were systematically diagnosed with stage I-III resectable NSCLC and from whom tumors were collected in tissue bank at the Medical University of Gdansk, Poland. The majority of patients were males, with squamous cell histology, smokers, older than 60 years (Table 1). Primary tumors were fresh-frozen at the time of surgery and stored at −80° C.

TABLE 1
Patients Characteristics

| Gender: | |
|---|---|
| Female | 62 |
| Male | 23 |
| Age: | |
| <60 | 26 |
| ≧60 | 59 |
| Smoking Status: | |
| Never | 3 |
| Ever | 82 |
| Histology: | |
| Sq. Cell Carcinoma | 51 |
| Adenocarcinoma | 21 |
| Mixed/other | 13 |
| Pathological Stage: | |
| I | 34 |
| II | 16 |
| III | 31 |
| IV | 4 |

Cell Culture, Drugs and MTT assay and transfection. Cell lines culture and MTT assay (3-[4,5-dimethylthiazol-2-yl]-2,5-diphenyltetrazolium bromide assay) was done as previously described. In brief, 15 NSCLC cell lines of those histologies were used: squamous cell carcinoma large-cell adenocarcinoma and bronchioloalveolar carcinoma Cell lines were treated with the IGF-1R TKI cyclolignan picropodophyllin (PPP) for 2 hrs and were and were analyzed with MTT after 4 days of incubation.

RNA, Primers, and Quantitative Real-Time RT-PCR. Total RNA was prepared from NSCLC cell lines and patients fresh frozen tumors using the RNAeasy kit (Qiagen). cDNA was transcribed from 1 μg of each sample using AffinityScript™ QPCR cDNA Synthesis Kit (Stratagene, La Jolla, Calif.). Quantitative Real-Time PCR were performed 1/20th of the cDNA reaction using the Brilliant® SYBR® Green QPCR Core Reagent Kit (Stratagene). Amplification data were analyzed by using GENEAMP 5700 SDS software, converted into cycle numbers at a set cycle threshold (Ct values) and quantified in relation to a standard. Human adult-lung RNA (Clontech Lab. Inc) was used as standard at 20, 4, 0.8, 0.16 ng in all the experiments. To normalize for the amount of input cDNA, the quantified relative amount of the generated product was divided by the amount generated for β-actin. Cycling conditions were 50° C. for 10 seconds and 95° C. for 10 minutes, followed by 46 cycles at 95° C. for 15 seconds and 60° C. for 1 minute. All samples were performed in triplicates. Primers were used as E-cadherin (forward) CGG GAA TGC AGT TGA GGA TC (SEQ ID NO:1), (reverse) AGG ATG GTG TAA GCG ATG GC (SEQ ID NO:2); ErbB3 (forward) GGGTTAGAGGAAGAGGATGTCAAC (SEQ ID NO;3), (reverse) GGGAGGAGGGAGTACCTTTGAG (SEQ ID NO:4); EGFR (forward) CCACCTGTGCCATCCAAAC (SEQ ID NO:5), (reverse) TCGTTGGACAGCCTTCAAGAC (SEQ ID NO:6) surviving and β-actin (forward) GAGCGCGGCTACAGCTT (SEQ ID NO:7), (reverse) TCCTTAATGTCACGCACGATTT (SEQ ID NO:8); IGF1R (forward) TGAAAGTGACGTCCTGCATTTC (SEQ ID NO:9), (reverse) GGTACCGGTGCCAGGTTATG (SEQ ID NO:10), surviving (forward) TCCACTGCCCCACTGAGAAC (SEQ ID NO:11), (reverse) TGGCTCCCAGCCTTCCA (SEQ ID NO:12).

### Example 2

### Results

Correlation between IGF-1R, Survivin, E-cadherin and ErbB3 in patients with NSCLC and in subset of patients with SCC. Following the experimental protocols set forth above, significant correlations were detected between IGF-1R and Survivin, between IGF-1R and E-cadherin, and between IGF-1R and ErbB3, in 85 patients with NSCLC (r=0.28, p=0.008; r=0.47, p<0.001; r=0.35,p=0.001, respectively) (Table 2).

TABLE 2
Correlations

| Gene | Survivin | EGFR | ERBB3 | IGF1R | ECADHER |
|---|---|---|---|---|---|
| Survivin | 1.000 | .166 | .040 | .286 | .466 |
| | — | .130 | .714 | .008 | .000 |
| | 85 | 85 | 85 | 85 | 85 |
| EGFR | .166 | 1.000 | .202 | .137 | .299 |
| | .130 | — | .064 | .211 | .005 |
| | 85 | 85 | 85 | 85 | 85 |
| ERBB3 | .040 | .202 | 1.000 | .351 | .405 |
| | .714 | .064 | — | .001 | .000 |
| | 85 | 85 | 85 | 85 | 85 |
| IGF1R | .286 | .137 | .351 | 1.000 | .276 |
| | .008 | .211 | .001 | — | .011 |
| | 85 | 85 | 85 | 85 | 85 |
| ECADHER | .466 | .299 | .405 | .276 | 1.000 |
| | .000 | .005 | .000 | .011 | — |
| | 85 | 85 | 85 | 85 | 85 |

Table 2 indicates r values (top number in each box), p value (middle value in each box) and # of patients (lower number).

The inventors next evaluated the expression of surviving in patients with SCC versus other histologies. They found that SCC histology associated significantly with Survivin and with IGF-1R expression (p=0.033, p=0.007; respectively).

Correlation between Survivin and activity of PPP in lung cancer cell lines. In 15 lung cancer lines, expression of survivin correlated with activity of the IGF-1R TKI, PPP (r=0.81, p<0.001).

All of the compositions and/or methods disclosed and claimed herein can be made and executed without undue experimentation in light of the present disclosure. While the compositions and methods of this invention have been described in terms of particular embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference:
U.S. Pat. No. 4,683,195
U.S. Pat. No. 4,683,202
U.S. Pat. No. 4,800,159
U.S. Pat. No. 4,883,750
U.S. Pat. No. 5,279,721
U.S. Pat. No. 7,371,378
U.S. Patent Publn. 2006/0140960
U.S. Patent Publn. 2007/0243194
U.S. Patent Publn. 2008/00669539
U.S. Patent Publn. 2008/0152649
U.S. Patent Publn. 2008/0181891
Bellus, J. Macromol. Sci. Pure Appl. Chem., A31(1): 1355-1376, 1994.
Burtrum et al., Cancer Res., 63:8912-8921, 2003.
Butler et al., Comparative Biochem. Physiol., 121:19, 1998.
Cancer: Principles and Practice of Oncology, 7^{th} Ed., 2004.
Chan et al., Science, 279:563, 1998.
Clinical Oncology, 3^{rd} Ed., 2004.
EPO No. 320 308
EPO No. 329 822
Fodor et al., Biochemistry, 30(33):8102-8108, 1991.
Freifelder, In: Physical Biochemistry Applications to Biochemistry and Molecular Biology, 2nd Ed. Wm. Freeman and Co., NY, 1982.
Frohman, In: PCR Protocols: A Guide To Methods And Applications, Academic Press, N.Y., 1990.
GB Appln. No. 2 202 328
Girnita et al. Cancer Res., 64(1):236-242, 2004.
Hacia et al., Nature Genet., 14:441-449, 1996.
Innis, et al., In: PCR Protocols. A guide to Methods and Application, Academic Press, Inc. San Diego, 1990.
Klapper et al., Endocrinol., 112:2215, 1983.
Kwoh et al., Proc. Natl. Acad. Sci. USA, 86:1173, 1989.
Macaulay, Br. J. Cancer, 65:311, 1992.
Ohara et al., Proc. Natl. Acad. Sci. USA, 86:5673-5677, 1989.
Pandini et al., Cancer Res., 5:1935, 1999.
PCT Appln. PCT/US87/00880
PCT Appln. PCT/US89/01025
Pease et al., Proc. Natl. Acad. Sci. USA, 91:5022-5026, 1994.
Pekonen et al., Cancer Res., 48:1343, 1998.
Pignon et al., Hum. Mutat., 3: 126-132, 1994.
Rinderknecht et al., Febs. Lett., 89:283, 1978.
Sambrook et al., In: Molecular cloning: a laboratory manual, 2^{nd} Ed., Cold Spring Harbor Laboratory Press, Cold Spring Harbor, N.Y., 1989.
Sepp-Lorenzino, Breast Cancer Res. Treatment, 47:235, 1998.
Shoemaker et al., Nature Genetics, 14:450-456, 1996.
Steller, et al., Cancer Res., 56:1762, 1996.
Walker et al., Nucleic Acids Res. 20(7):1691-1696, 1992.
Webster et al., Cancer Res., 56:2781, 1996.
PCT Appln. WO 2002/53596
PCT Appln. WO 2002/92599
PCT Appln. WO 2003/100008
PCT Appln. WO 2003/106621
PCT Appln. WO 2003/39538
PCT Appln. WO 2003/59951
PCT Appln. WO 2004/71529
PCT Appln. WO 2004/83248
PCT Appln. WO 2004/87756
PCT Appln. WO 88/10315
PCT Appln. WO 89/06700
PCT Appln. WO 90/07641
PCT Appln. WO0208436
PCT Appln. WO0231168
PCT Appln. WO0285287
PCT Appln. WO0333029
Wu et al., Genomics, 4:560-569, 1989.
Xie et al., Cancer Res., 59:3588, 1999.

1. A method of predicting response of a cancer cell tissue to IGF-1R treatment comprising:
(a) assessing the expression of one or more of survivin, ErbB3 and E-cadherin in said cancer cell tissue; and
(b) predicting a favorable response if one or more of survivin, ErbB3 and E-cadherin is detected in said cancer cell tissue.

2. The method of claim 1, wherein the expression of two or more of survivin, ErbB3 and E-cadherin is detected.

3. The method of claim 3, wherein the expression of all three of survivin, ErbB3 and E-cadherin are detected.

4. The method of claim 1, further comprising assessing the expression of IGF-1R in said cancer cell tissue.

5. The method of claim 1, wherein expression of one or more of survivin, ErbB3 and E-cadherin is elevated with respect to average expression in a normal population.

6. The method of claim 6, wherein expression of all three of survivin, ErbB3 and E-cadherin are elevated with respect to average expression in a normal population.

7. The method of claim 1, further comprising treating said cancer cell tissue with an inhibitor of IGF-1R.

8. The method of claim 7, wherein said inhibitor is TKI.

9. The method of claim 7, further comprising, following treating, assessing the expression of one or more of survivin, ErbB3 and E-cadherin in said cancer cell tissue.

10. The method of claim 1, further comprising obtaining said cancer cell tissue by biopsy of a tumor from a subject.

11. The method of claim 10, further comprising making a treatment decision for said subject based on the outcome of step (b).

12-13. (canceled)

14. The method of claim 1, wherein said cancer tissue is lung cancer tissue, prostate cancer tissue, pancreatic cancer tissue, gastric cancer tissue, esophageal cancer tissue, colon cancer tissue or breast cancer tissue.

15. The method of claim 14, wherein said lung cancer tissue is non-small cell lung cancer tissue.

16. A method of predicting outcome of an IGF-1R cancer treatment in a subject comprising:
(a) assessing the expression of one or more of survivin, ErbB3 and E-cadherin in cancer cells from said subject;
(b) treating a subject with an IGF-1R cancer treatment if expression of one or more of survivin, ErbB3 and E-cadherin is detected, or treating a subject with a non-IGF-1R cancer treatment if expression of survivin, ErbB3 or E-cadherin is not detected.

17. The method of claim 16, wherein the expression of two or more of survivin, ErbB3 and E-cadherin is detected.

18. The method of claim 16, wherein the expression of all three of survivin, ErbB3 and E-cadherin are detected.

19. The method of claim 16, further comprising assessing the expression of IGF-1R in cancer cells from said subject.

20. The method of claim 16, wherein expression of one or more of survivin, ErbB3 and E-cadherin is elevated with respect to average expression in a normal population.

21. The method of claim 16, wherein expression of all three of survivin, ErbB3 and E-cadherin are elevated with respect to average expression in a normal population.

22. (canceled)

23. The method of claim 16, wherein said IGF-1R cancer treatment is TKI.

24. The method of claim 16, further comprising obtaining said cancer cells by a first biopsy of a tumor from said subject.

25. The method of claim 24, further comprising obtaining, after step (b), additional cancer cells by a second biopsy of said tumor from said subject, and assessing the expression of one or more of survivin, ErbB3 and E-cadherin in cancer cells of said second biopsy.

26. The method of claim 16, wherein said a non-IGF-1R cancer treatment comprises chemotherapy, radiotherapy and/or surgery.

27-28. (canceled)

29. The method of claim 16, wherein said cancer tissue is lung cancer tissue, prostate cancer tissue, pancreatic cancer tissue, gastric cancer tissue, esophageal cancer tissue, colon cancer tissue or breast cancer tissue.

30. The method of claim 29, wherein said lung cancer tissue is non-small cell lung cancer tissue.


SEQUENCE LISTING (12 sequences)

SEQ ID NO: 1 - DNA, 20 bp, Artificial Sequence
  Synthetic primer
cgggaatgcagttgaggatc

SEQ ID NO: 2 - DNA, 20 bp, Artificial Sequence
  Synthetic primer
aggatggtgtaagcgatggc

SEQ ID NO: 3 - DNA, 24 bp, Artificial Sequence
  Synthetic primer
gggttagaggaagaggatgtcaac

SEQ ID NO: 4 - DNA, 22 bp, Artificial Sequence
  Synthetic primer
gggaggagggagtacctttgag

SEQ ID NO: 5 - DNA, 19 bp, Artificial Sequence
  Synthetic primer
ccacctgtgccatccaaac

SEQ ID NO: 6 - DNA, 21 bp, Artificial Sequence
  Synthetic primer
tcgttggacagccttcaagac

SEQ ID NO: 7 - DNA, 17 bp, Artificial Sequence
  Synthetic primer
gagcgcggctacagctt

SEQ ID NO: 8 - DNA, 22 bp, Artificial Sequence
  Synthetic primer
tccttaatgtcacgcacgattt

SEQ ID NO: 9 - DNA, 22 bp, Artificial Sequence
  Synthetic primer
tgaaagtgacgtcctgcatttc

SEQ ID NO: 10 - DNA, 20 bp, Artificial Sequence
  Synthetic primer
ggtaccggtgccaggttatg

SEQ ID NO: 11 - DNA, 20 bp, Artificial Sequence
  Synthetic primer
tccactgccccactgagaac

SEQ ID NO: 12 - DNA, 17 bp, Artificial Sequence
  Synthetic primer
tggctcccagccttcca